(19) United States
(12) Patent Application Publication
(10) Pub. No.: US 2008/0267977 A1
(43) Pub. Date: Oct. 30, 2008

(54) Combined immunological agent and sensitizing agent for the treatment of cancer

(75) Inventors: Georg H.M. Fey, Neunkirchen (DE); Julia Stieglmaier, Erlangen (DE); Michael Schwenkert, Nuremberg (DE); Michael Schwemmlein, Erlangen (DE)

Correspondence Address: PERKINS COIE LLP, P.O. BOX 1208, SEATTLE, WA, 98111-1208, US

(73) Assignee: Friedrich-Alexander University of Erlangen-Nuremberg, Erlangen (DE)

(21) Appl. No.: 11/796,517

(22) Filed: Apr. 26, 2007

(51) Int. Cl. A61K 39/44; A61K 31/19; A61P 35/00; A61K 38/13

(52) U.S. Cl. 424/174.1; 514/557; 514/9

(57) ABSTRACT

A method and kit for inhibiting the proliferation of cancer cells are disclosed, based on a combination of an apoptosis-inducing immunologic agent, and a sensitizing agent. When used in cancer therapy, the two agents in combination enhance the anti-cancer treatment efficacy obtained with the immunologic agent or the sensitizing agent alone, by a supraadditive amount.

### FIELD OF THE INVENTION

The invention is directed to anticancer treatment, and in particular to inhibition of tumor growth or cancer-cell proliferation, by treatment with an immunological agent effective to induce cancer-cell apoptosis and a sensitizing agent.

### BACKGROUND

Although many cancers can be cured by surgical resection, chemotherapy is often used as an adjunct to surgical therapy, and it is widely used in the treatment of inoperable or metastatic malignancy. In view of the continuing high number of deaths each year resulting from cancer, a continuing need exists to identify effective and relatively nontoxic therapeutic regimens for use in anticancer treatment.

Many effective chemotherapeutic agents have been identified over the past few decades, and these are generally grouped into several categories on the basis of their mechanism of action. Combined-therapy treatments have become more common, in view of the perceived advantage of attacking the disease via multiple avenues. In practice, however, many such combinations do not provide even simple additivity of therapeutic effects.

Ideally, a combined-drug approach for cancer treatment should provide a significant boost in efficacy and/or a significant reduction in undesired side effects, due to a reduced dose of the more toxic component and/or a reduction in the development of drug-resistance in the cancer being treated. Particularly desirable are combination therapies which produce therapeutic results that are supraadditive or synergistic in nature relative to the effects of the individual agents, with minimal exacerbation of side effects.

### SUMMARY

The invention includes, in one aspect, a method for inhibiting the proliferation of cancer cells, by the steps of (a) exposing the cells to an immunological agent capable of binding specifically to an antigen expressed on the surface of the cancer cells, in an amount of immunological agent, when given alone, that is effective to inhibit proliferation of cancer cells by inducing apoptosis in the cells, and (b) either proceeding, following, or concomitantly with step (a), exposing the cells to a sensitizing agent selected from cyclosporin A or an analog thereof, or valproic acid or an analog thereof. The amount of sensitizing agent to which the cells are exposed is effective to potentiate the anti-cancer effect of the immunologic agent, as evidenced by a level of inhibition of cancer-cell proliferation produced by exposing the cells to both agents that is supraadditive relative to the sum of the inhibitions of cancer-cell proliferation observed by exposing the cells to the immunological agent alone and to the sensitizing agent alone.

The apoptosis-inducing immunological agent may be, for example an antibody alone or an antibody conjugated to a cell toxin, such as a protein toxin. An exemplary toxin includes Pseudomonas exotoxin A (ETA), or an antibody conjugated to another active agent, such as a small-molecule anti-cancer agent.

Exemplary sensitizing agents include cyclosporin A and analogs thereof, such as NIM811, UNIL025, and PKF220-384, and valproic acid and analogs thereof, such as 2-propyl-4-pentynoic acid.

For use in treating cancer in a subject, step (a) in the method includes administering to the subject, an amount of the apoptosis-inducing agent, e.g., immunological agent that, when given alone, would be effective to inhibit proliferation of cancer cells in the subject, and step (b) includes administering to the subject, an amount of the sensitizing agent effective to potentiate the anti-cancer effect of the immunologic agent, as evidenced by a level of inhibition of cancer-cell proliferation produced by administering both agents to the subject that is supraadditive relative to the sum of the inhibitions of cancer-cell proliferation observed by administering the immunological agent alone and the sensitizing agent alone.

For use in treating a B-cell leukemia in a subject, the apoptosis-inducing immunologic agent may include a CD19 antibody or a CD19 antibody conjugate of an active moiety selected from the group consisting of a toxin or a B-cell receptor ligand. For use in treating a melanoma, the immunological agent may include an MCSP-directed antibody.

Also disclosed is a method for enhancing the anti-cancer treatment efficacy of an immunological agent capable, when administered to a subject with a given cancer, of binding specifically to an antigen expressed on the surface of cells of the cancer, to inhibit proliferation of the cells by inducing cell apoptosis. The enhancement is achieved by administering to the subject, before, during, or after administering the immunological agent, a sensitizing agent selected from cyclosporin A or an analog thereof, or valproic acid or an analog thereof, in an amount of sensitizing agent effective to potentiate the anti-cancer effect of the immunologic agent, as evidenced by a level of inhibition of cancer-cell proliferation produced by administering both agents to the subject that is supraadditive relative to the sum of the inhibitions of cancer-cell proliferation observed by administering the immunological agent alone and the sensitizing agent alone.

In another aspect, the invention includes a kit for use in treating a cancer in a subject. The kit includes (a) a dose of an immunological agent capable of binding specifically to an antigen expressed on the surface of cells of the cancer, effective in amount to inhibit proliferation of cancer cells in the subject by inducing cell apoptosis, and (b) a dose of a sensitizing agent selected from cyclosporin A or an analog thereof, or valproic acid or an analog thereof, effective in amount to potentiate the anti-cancer effect of the immunologic agent, as evidenced by a level of inhibition of cancer-cell proliferation produced by administering both agents to the subject that is supraadditive relative to the sum of the inhibitions of cancer-cell proliferation observed by administering the immunological agent alone and the sensitizing agent alone.

In a related embodiment, the invention includes a kit for use in treating a cancer in a subject. The kit includes (a) an immunological agent that, when administered to the subject in a therapeutic dose, is effective to inhibit proliferation of cancer cells in the subject by inducing cell apoptosis, and (b) a product insert having one set of directions for using the immunological agent in monotherapy, by administering the immunological agent to a subject at a therapeutic dose, and another set of directions for potentiating the anti-cancer effect of the immunological agent, when administered to the subject at a therapeutic dose, by administering to the subject, before, during, or after administration of the immunological agent, a sensitizing agent selected from cyclosporin A or an analog thereof, or valproic acid or an analog thereof, in amount of sensitizing agent effective to potentiate the anti-cancer effect of the immunological agent, as evidenced by a level of inhibition of cancer-cell proliferation produced by administering both agents to the subject that is supraadditive relative to the sum of the inhibitions of cancer-cell proliferation observed by administering the immunological agent alone and the sensitizing agent alone.

The therapeutic dose of the immunological agent to be administered, in accordance with the product insert, may be less for the combination therapy than for the monotherapy.

The invention is further directed to the use of an immunological agent capable of binding specifically to an antigen expressed on the surface of cancer cells to inhibit proliferation of cancer cells, by inducing apoptosis in the cells, and a sensitizing agent selected from cyclosporin A or an analog thereof, or valproic acid or an analog thereof, in the manufacture of a medicament for the treatment of cancer.

Also disclosed is a sensitizing agent selected cyclosporin A or an analog thereof, or valproic acid or an analog thereof, in the manufacture of a medicament for treating cancer in a subject who is being treated with an immunological agent capable of binding specifically to an antigen expressed on the surface of cancer cells to inhibit proliferation of cancer cells, by inducing apoptosis in the cells, for the purpose of enhancing the anti-cancer efficacy of the immunological agent in the subject.

These and other objects and features of the invention will become more fully apparent when the following detailed description of the invention is read in conjunction with the accompanying drawings.

### BRIEF DESCRIPTION OF THE DRAWINGS

FIG. 1A is a schematic representation of the recombinant scFv CD19:ETA immunotoxin. The tags are: STREP tag; 6×histidine tag; V_{L} and V_{H}, variable region light and heavy chains of the CD19-specific scFv; linker L, flexible linkers consisting of glycine and serine residues; ETA, truncated Exotoxin A fragment consisting of domains II and III of the Pseudomonas toxin; KDEL, ER retention motif;

FIG. 1B is a schematic representation of the recombinant scFv MCSP:ETA immunotoxin. The tags are: STREP tag; 6×histidine tag; V_{L} and V_{H}, variable region light and heavy chains of the MCSP-specific scFv; (G₄S)₄, a 20 amino acid linker composed of glycine and serine residues.

FIG. 2A illustrates that CD19:ETA induces apoptosis in antigen-positive cells. CD19-positive Nalm-6, Reh and Namalwa cells were treated with a single dose of 500 ng/ml CD19:ETA alone or in presence of 20-fold molar excess of the parental antibody 4G7. After 48 h cells were stained with Annexin V and PI.

FIG. 2B shows the results of treating Nalm-6, Reh and Namalwa cells with a single dose of 1 μg/ml CD19:ETA′ in the presence or in the absence of a 10-fold molar excess of the parental antibody 4G7. The cells were analyzed for cleavage of PARP by Western blot.

FIGS. 3A-3D show that MCSP:ETA′-KDEL induces apoptosis in MCSP-positive A2058 and A375M melanoma cells that can be blocked by the parental antibody 9.2.27. A2058 cells (3A) and A375M cells (3B) were treated with single doses of 1 μg/ml MCSP:ETA′-KDEL or 1 μg/ml MCSP:ETA′-KDEL and 22 μg/ml 9.2.27 respectively. Cells were stained with Annexin V and propidium iodide (PI) at the indicated time points and analyzed by flow-cytometry. Numbers in the “bottom right quadrant” of each dot blot represent the percentage of cells in early apoptotic stage (Annexin V-positive and PI-negative). The data is representative of three separate experiments. A2058 cells (3C) and A375M cells (3D) were treated with single doses of 1 μg/ml MCSP:ETA′-KDEL and analyzed for cleavage of poly ADP-ribose polymerase (PARP) by Western transfer experiments. The specific cleavage product of 85 kDa, indicated by the arrow, was only detectable in MCSP:ETA′-KDEL treated samples; lane 1, PBS treated cells; lane 2, MCSP:ETA′-KDEL treated cells.

FIGS. 4A and 4B show that VPA and CsA sensitize cells to induction of apoptosis by CD19:ETA. (4A) Nalm-6, Reh and SEM cells were left untreated (white bars) or were either treated with a single dose of 100 ng/ml CD19:ETA′ (bright grey bars), 150 μg/ml (Nalm-6) or 100 μg/ml (Reh, SEM) VPA (dark grey bars), respectively, or with a combination of both agents (black bars). After 72 h cells were stained with Annexin V and PI. Bars represent mean values from five independent experiments. (4B) Cells were left untreated (white bars) or were either treated with a single dose of 100 ng/ml CD19:ETA′ (bright grey bars), 6 μM (Nalm-6, Reh) or 10 μM (SEM) CsA (dark grey bars), respectively, or with a combination of both agents (black bars). After 48 h cells were stained with Annexin V and PI. Bars represent mean values from four (Reh) or five (Nalm-6, SEM) experiments. Standard deviations are indicated by error bars. An asterisk indicates p values≦0.005, two asterisks indicate p values≦0.0005. P values are given for differences in apoptosis induction between single-agent treatment and combination treatment.

FIGS. 5A and 5B demonstrate the synergistic cytoxic effect of MCSP:ETA′-KDEL and CyclosporinA (CsA). A2058 cells (5A) were treated with single doses of CsA (black) in varying concentrations, CsA in combination with a single dose of 100 ng/ml MCSP:ETA′-KDEL (grey) and CsA in combination with a single dose of 220 ng/ml 9.2.27 (white) for 72 h Primary melanoma cells from patient #4 (5B) were treated with single doses of 1 μg/ml MCSP -ETA′-KDEL (white), 5 μg/ml and 10 μg/ml CsA (black) respectively or both agents in combination (grey) for 48 h. Cells were evaluated for percentage of cell death by propidium iodide staining of nuclei and flow-cytometry. Percentage specific cell death was considered as percentage cell death above background. Data points are mean values from three independent experiments and standard deviations are indicated by error bars. Indicated values are calculated cooperative indices (cᵢ) of the two agents MCSP:ETA′-KDEL and CsA.

### DETAILED DESCRIPTION OF THE INVENTION

### I. Definitions

An “immunologic agent capable of binding specifically to an antigen expressed on the surface of the cancer cells” refers to any agent capable of binding specifically, i.e., with antigen specificity, to an antigen expressed on the surface of a cancer cell. An exemplary immunological agent is an antibody alone or an antibody conjugated to a cell toxin, such as a protein toxin, or an antibody conjugated to a small molecule chemotherapeutic agent.

An “antibody,” as used herein, encompasses an immunoglobulin molecule comprised of four polypeptide chains, two heavy (H) chains and two light (L) chains inter-connected by disulfide bonds, and antigen-binding fragments and variants thereof, as considered below. Each chain in an antibody typically consists of a variable portion, denoted V_{H} and V_{L} for variable heavy and variable light portions, respectively, and a constant region, denoted C_{H} and C_{L} for constant heavy and constant light portions, respectively.

The term “antibody” also encompasses immunologically specific antibody fragments, such as (i) an Fab fragment, which is a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) an Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). In particular, although the two domains of the Fv fragment, V_{L} and V_{H}, are coded for by separate genes, they can be joined by recombinant methods, by a synthetic linker that enables them to be made as a single protein chain in which the V_{L} and V_{H} regions pair to form monovalent molecules known as single chain variable fragment or scFv antibodies; see e.g., Bird et al. (1988) Science 242:423-426; and Huston et al. (1988) Proc. Natl. Acad. Sci. USA 85:5879-5883), and the term antibody lacking an Fc fragment also encompasses antibodies having this scFv format.

The term “antibody” also encompasses the antibody moiety, e.g., portion, of an immunologic agent composed of an antibody moiety conjugated to a second moiety, such as a protein toxin or small-molecule anti-tumor agent.

The term “recombinant antibody”, as used herein, is intended to include antibodies that are prepared, expressed, created or isolated by recombinant means, such as antibodies expressed using a recombinant expression vector transfected into a host cell.

A “glycine/serine” linker refers to a linear polypeptide chain composed substantially, e.g., at least 80%, and preferably entirely of glycine and serine amino acid residues.

The three-letter and one-letter amino acid abbreviations and the single-letter nucleotide base abbreviations used herein are according to established convention, as given in any standard biochemistry or molecular biology textbook.

An “apoptosis-inducing agent” refers to an agent that acts to inhibit cancer-cell proliferation or tumor growth, at least in part, by inducing apoptosis or programmed cell death in cancer cells. The apoptosis inducing moiety in an immunological agent may reside in the immunological moiety alone, e.g., antibody, or in an active moiety conjugated to the antibody.

A “sensitizing agent,” as used herein, refers to cycloporin A (CSA), valproic acid (VPA), and analogs of CSA and VPA that have the ability, like CSA or VPA themselves, to potentiate the ability of immunological agents to inhibit cancer-cell proliferation by inducing cell apoptosis.

An agent is said to “inhibit the proliferation of cancer cells” if the proliferation of cells in the presence of the agent is less than that observed in the absence of the agent. That is, proliferation of the cells is either slowed or halted in the presence of the agent. Inhibition of cancer-cell proliferation may be evidenced, for example, by reduction in the number of cells or rate of expansion of cells, reduction in tumor mass or the rate of tumor growth, or increase in survival rate of a subject being treated.

### II. Immunological Agent

The immunological agent employed in the invention is designed to react immuno-specifically with an antigen found on the surface of cancer cells, and through its interaction with the cell, induce cell apoptosis. The immunological agent may include an antibody alone, or an antibody moiety conjugated to an active moiety which itself has anti-cancer or anti-tumor properties.

A. The Antibody Moiety of the Immunological Agent

The antibody in the immunological agent is immunoreactive against a cell-surface antigen present on the surface of target cancers cells. Examples of immunological agents, and associated cancers that are targets for the agent, include agents whose immunological moiety is designed for targeting CD19 or CD20, for treating of B-lineage leukemias, such as chronic lymphoblastic leukemia (CLL) and non-Hodgkin-lymphomas (NHL), agents targeting CD22, for treating hairy cell leukemias, agents targeting CD25, CD7, CD64, or CD33, for treating various haematological malignancies expressing CD25, CD7, CD64, or CD33, respectively, agents targeting Melanoma associated Chondrotin Sulfate Proteoglycan (MCSP) antigen, for treating malignant melanomas, agents targeting a Lewis Y Antigen, for treating adenocarcinomas, and agents targeting IL13 receptor or EGF receptor (EGFR), CD52, HER2/neu, and VEGF, for treating a variety of tumors known to express these antigens.

Methods for preparing immunological agents having a desired antigen specificity can be prepared in accordance with known methods, such as those detailed in Example 1 below for the production of single-chain Fragment variable (scFv) antibodies described below for producing an immunological agent designed for target cancer cells with CD19 surface antigen or MCSP antigen.

The antibody of the present invention is preferably a human or humanized antibody suitable for human therapy. Humanized antibodies can be prepared based on the sequence of a murine monoclonal antibody prepared according to conventional monoclonal antibody techniques. DNA encoding the heavy and light chain immunoglobulins can be obtained from the murine hybridoma of interest and engineered to contain human immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

More generally, humanized antibodies may be prepared by (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et a., Proc. Natl. Acad. Sci. 81: 6851-5 (1984); Morrison et al., Adv. Immunol. 44: 65-92 (1988); Verhoeyen et al., Science 239: 1534-1536 (1988); Padlan, Molec. Immun. 28: 489-498 (1991); Padlan, Molec. Immun. 31: 169-217 (1994), and U.S. Pat. Nos. 5,585,089, 5,693,761 and 5,693,762 all of which are hereby incorporated by reference in their entirety.

Human monoclonal antibodies directed against CD19 can be produced using transgenic or transchromosomic mice carrying parts of the human immune system rather than the mouse system. These transgenic and transchromosomic mice include mice referred to herein as the HuMAb Mouse® and KM Mouse® respectively, and are collectively referred to herein as “human Ig mice.” The HuMAb Mouse® (Medarex®, Inc.) contains human immunoglobulin gene miniloci that encode unrearranged human heavy (mu and gamma) and kappa light chain immunoglobulin sequences, together with targeted mutations that inactivate the endogenous mu. and kappa. chain loci (see e.g., Lonberg, et al. (1994) Nature 368(6474): 856-859). Accordingly, the mice exhibit reduced expression of mouse IgM or .kappa., and in response to immunization, the introduced human heavy and light chain transgenes undergo class switching and somatic mutation to generate high affinity human IgG.kappa. monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546).

The preparation and use of the HuMab Mouse®, and the genomic modifications carried by such mice, is further described in Taylor, L. et al. (1992) Nucleic Acids Research 20:6287-6295; Chen, J. et al. (1993) International Immunology 5:647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6:579-591; and Fishwild, D. et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al. preferably.

One exemplary antibody is a scFv antibody prepared against CD19 or MCSP, as detailed in Example 1 below.

B. The Active Moiety in the Immunological Agent

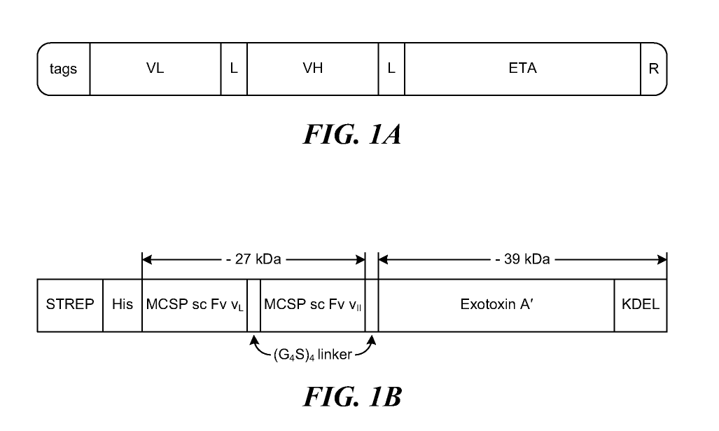

As noted above, the immunologic agent may include an active moiety conjugated to an antibody moiety. The active moiety in the immunological agent may be a peptide cytotoxin, such as Pseudomonas Exotoxin A (ETA), and specifically, the cytotoxic domains II, IB, and III of the toxin (Pastan, I., et al., J Biol Chem, 264:15157-15160 (1989). Further, the toxin may be modified, at its C-terminal end, to include a KDEL sequence, which is known to improve the retrograde transport through the trans-golgi network and enhance the cytotoxicity of ETA-derived immunotoxins (Seetharam, S., et al., J Biol Chem, 266:17376-17381 (1991). Examples 1A and 1B below detail the construction of immunologic agents containing a modified ETA peptide conjugated to either an anti-CD19 scFv antibody (FIG. 1A) or an anti-MCSP scFv antibody (FIG. 1B). Other cytotoxic agents suitable as the active moiety in the immunological agents are the toxic peptides saporin, gelonin, ricin, diptheria toxin, trichosanthin, and pokeweed antiviral proteins.

An advantage of an immunological agent composed of an antibody moiety conjugated to an active moiety is that the agent may be produced as a single polypeptide by recombinant techniques, such as described in Example 1 below, where the two moieties are coupled through a well-defined linkage, such as a glycine-serine linker. However, the active moiety may also be a small-molecule, non-peptide toxin, such as a chemotherapeutic agent, e.g., a cisplatin, a taxol, or an etoposide agent, that is coupled to the antibody moiety by standard chemical coupling methods, e.g., employing a divalent linker. In one embodiment, the antibody moiety terminates in reaction-rich peptide chain, such as one containing multiple carboxyl, amine or sulhydral groups to which the chemotherapeutic agent may be selectively coupled.

C. The Immunological Agent as an Apoptosis-Inducing Agent

The immunologic agent employed in the invention inhibits cancer-cell growth through a mechanism involving programmed cell death, or apoptosis. One test for demonstrating that the agent's ability to inhibit cancer-cell growth is attributable to apoptosis is by an Annexin V and PI double staining method in a flow cytometry analysis. This method, which detects cells in an early apoptotic stage, is detailed in Example 2A for the CD19:ETA, and in Example 2B for the MCSP:ETA agent.

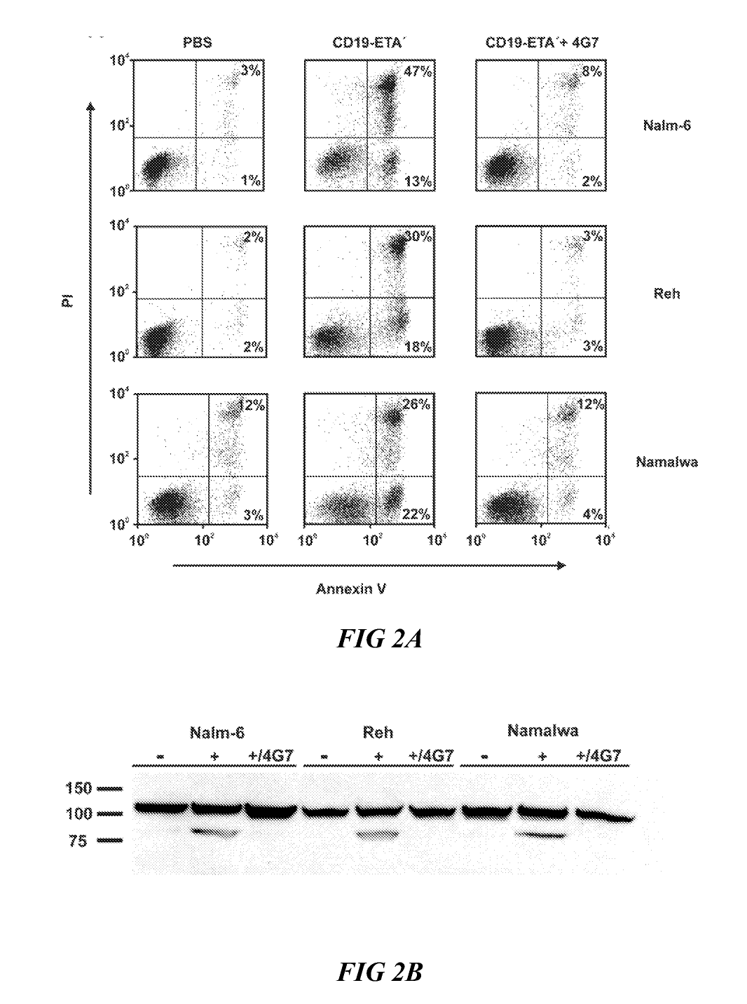

In Example 1A, double-staining analysis of CD19-positive Nalm-6, Reh, and Namalwa cells treated with PBS (control), CD19:ETA, and CD19:ETA plus a 20-fold molar excess of parental CD19 antibody (4G7) is seen in the three columns of FIG. 2A. Numbers in the bottom right quadrant of each plot represent the percentage of cells in early apoptosis (Annexin V-positive and PI-negative). Numbers in the upper right quadrant represent the percentage of dead cells (Annexin V-positive and PI-positive). It can be seen that the CD19:ETA immunological agent was active in inducing apoptosis, as a mechanism of cell death.

Another characteristic feature of an apoptotic mechanism of action is agent-induced cleavage of poly(ADP-ribose) polymerase (PARP). In the study reported in Example 2A, Nalm-6, Reh, and Namalwa cells were treated with CD19:ETA for 24 hours, and cleavage of PARP was analyzed by Western blot, with the results shown in FIG. 2B. As seen, CD19:ETA treatment induced in PARP cleavage in all three cells, and this cleavage was largely prevented by adding a 10-fold molar excess of the parental 4G7 CD19 antibody.

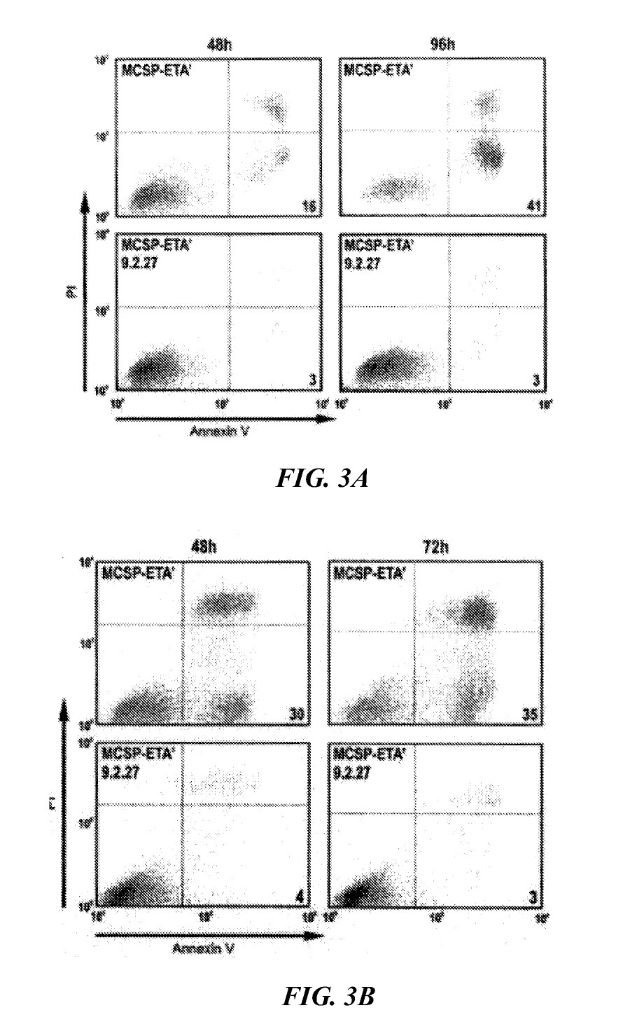

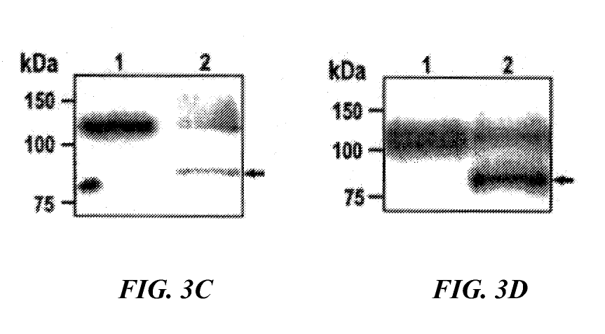

Similar results demonstrating an apoptotic mechanism for the MCSP:ETA agent are shown in FIGS. 3A-3B, and discussed below in Example 2B, again indicating the various in vitro cell tests that may be employed to demonstrate that the immunologic agent employed in a cancer-treatment method is acting, at least in part, through mechanism involving induction of apoptosis in the exposed cells.

### III. Sensitizing Agent

In accordance with the invention, it has been discovered that certain sensitizing agents are able to potentiate the ability of immunological agents described above to inhibit cancer-cell growth. In particular, the level of inhibition of cancer-cell proliferation produced by exposing the cells to both the immunological agent and sensitizer is supraadditive relative to the sum of the inhibitions of cancer-cell proliferation observed by exposing the cells to the immunological agent alone and to the sensitizer alone.

One exemplary group of sensitizing agents are the cyclosporins, a family of immunospressive compounds isolated from fermentation broths of various fungal species including Tolypocladium inflatum and Cylindrocarpon lucidum. The generic structure of the class of cyclosporins has been established as a cyclic peptide which contains 11 amino acids. Cyclosporin A (CsA) contains several N-methylated amino acids and one novel amino acid “MeBMT” designated as the 1 “C-9 amino acid”. This novel amino acid is located in position 1 and has been found to be important for the biological activity of cyclosporin. It has been found that replacing the double bond of the “C-9 amino acid” (MeBMT) with a hetero atom such as S and O decreases the toxicity of the parent cyclosporin, but the analog contains substantial activity in the various assays in which cyclosporin A expresses immunosuppressive activity is also exhibited.

Structural analogs in the cyclosporin family, including structural analogs of CsA that are contemplated for use in the present invention include those disclosed in U.S. Pat. No. 7,141,648 for Synthesis of cyclosporin analogs; U.S. Pat. No. 6,809,077, for Cyclosporin analogs for the treatment of autoimmune diseases; U.S. Pat. No. 5,236,899 for 6-position cyclosporin a analogs as modifiers of cytotoxic drug resistance; U.S. Pat. No. 5,227,467, for Immunosuppressive fluorinated cyclosporin analogs; U.S. Pat. No. 5,214,130, for Synthesis of novel immunosuppressive cyclosporin analogs with modified amino acids at position-8; U.S. Pat. No. 5,122,511, for Immunosuppressive cyclosporin analogs with modified amino acids at position-8; U.S. Pat. No. 4,914,188 for Novel 6-position cyclosporin analogs as non-immunosuppressive antagonists of cyclosporin binding to cyclophilin; U.S. Pat. No. 4,885,276 for Cyclosporin analogs with modified “C-9 amino acids”; and U.S. Pat. No. 4,798,823, for New cyclosporin analogs with modified “C-9 amino acids”, all of which are incorporated by reference herein.

Another exemplary class of sensitizing agents is valproic acid (VPA) and structural analogs thereof, such as propyl-4-yn-valproic acid(2-propyl-4-pentynoic acid). VPA, a member of the short chain fatty acids, is widely used for treatment of various kinds of epilepsy. Recently, VPA has been identified as an inhibitor of histone deacetylases (HDACs) able to induce differentiation of transformed cells (Goettlicher, M., Embo J., 20:6969-6978 (2001)). Furthermore, by inhibiting deacetylation of histones and thereby restoring expression of genes involved in tumor suppression and cell cycle regulation, VPA has been demonstrated to block proliferation and induce apoptosis of human leukemia cells, including B-cell precursor leukemia cell lines (Kawagoe, R., et al., Leuk Res, 26:495-502 (2002); Sakajiri, S., et al., Exp Hematl, 33:53-61 (2005); Einsiedel, H. G., Leukemia, 20:1435-1346 (2006).

Structural analogs of VPA that are contemplated for use in the present invention are those disclosed in U.S. Pat. No. 6,555,585 for Use of derivatives of valproic acid and 2-valproenic acid amides for the treatment of mania in bipolar disorder; U.S. Pat. No. 6,458,840, for Use of valproic acid analog for the treatment and prevention of migraine and affective illness; U.S. Pat. No. 6,323,365, for Active derivative of valproic acid for the treatment of neurological and psychotic disorders and a method for their preparation; U.S. Pat. No. 6,313,106, for Phospholipid derivatives of valproic acid and mixtures thereof; U.S. Pat. No. 6,268,396 for Use of valproic acid analog for the treatment and prevention of migraine and affective illness; U.S. Pat. No. 5,585,358, for Derivatives of valproic acid amides and 2-valproenoic acid amides, method of making and use thereof as anticonvulsant agents U.S. Pat. No. 5,440,023, for Method for making valproic acid derivatives, U.S. Pat. No. 5,162,573, for Valproic and (E)-2-valproenoic acid derivatives, and pharmaceutical compositions therefrom, U.S. Pat. No. 4,595,695, for 1′-ethoxycarbonyloxyethyl ester of valproic acid, its preparation and pharmaceutical compositions containing it, and U.S. Pat. No. 4,442,124 for Valproic acid ester with antiepileptic and anticonvulsant activity and pharmaceutical compositions therefrom, all of which are incorporated herein by reference.

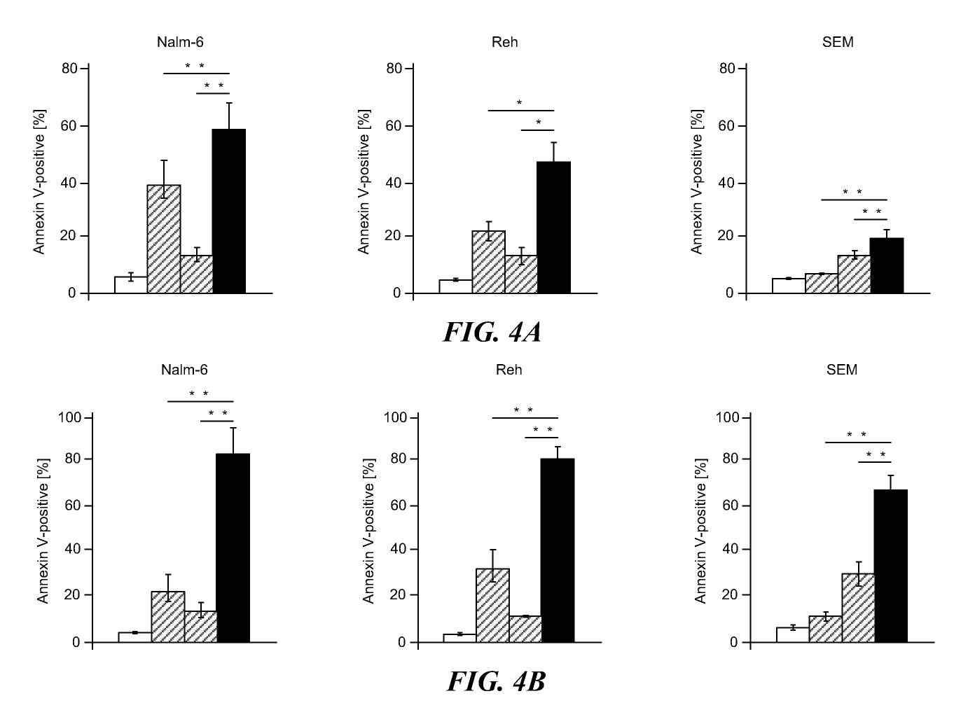

The ability of CsA and VPA to potentiate the inhibitory effect of several immunological agents is detailed in the studies reported in Example 3. In Example 3A, the effect of combined treatment with CD19:ETA agent plus CSA or VPA is examined, with the results shown in FIGS. 4A and 4B. In both figures, white bars indicate untreated Nalm-6, Reh and SEM cells. Cell treatment involved a single dose of 100 ng/ml CD19:ETA′ (bright grey bars), 150 μg/ml (Nalm-6) or 100 μg/ml (Reh, SEM) VPA (dark grey bars), respectively, or with a combination of both agents (black bars). After 72 h cells were stained with Annexin V and PI. In 5B, the same treatment was applied, but with CsA treatment at 6 μM (Nalm-6, Reh) or 10 μM (SEM) CsA (dark grey bars), respectively, or with a combination of both agents (black bars). As seen in the figures, CD19ETA with CsA and VPA produced a degree of cell death that is greater than the sum of the effects seen with either agent alone.

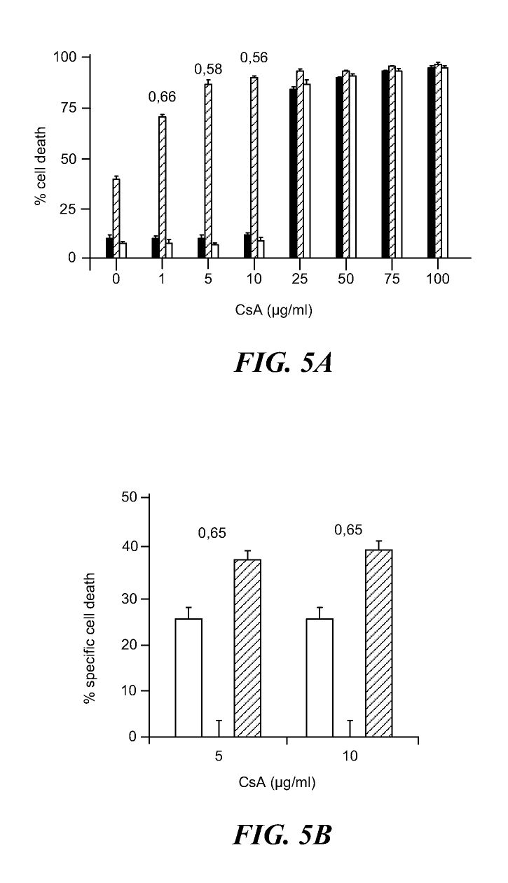

FIG. 5A illustrates the synergistic cytotoxic effect of MCSP:ETA in combination with CsA, as a function of increasing concentrations of CsA. As seen, at levels at which CsA was itself (black bars) was not highly toxic, i.e., at 10 μg/ml CsA and below, CsA produced a severalfold potentiation of the MCSP:ETA agent (white bars, given alone; grey bars, in combination with CsA). These results are discussed below in Example 3B.

IV. Combination Therapy with Immunological Agent and Sensitizing Agent

For use in treating a subject with cancer, in accordance with the present invention, the cancer must be one that responds to an immunological agent directed against a target cancer-cell antigen. Examples of immunological agents, and associated cancers which are targets for the agent, include agents whose immunological moiety is designed for targeting CD19 and CD20, for treating of B-lineage leukemias, such as chronic lymphoblastic leukemia (CLL) and non-Hodgkin-lymphomas (NHL), agents targeting CD22, for treating hairy cell leukemias, agents targeting CD25, CD7, CD64, and CD33, for treating various haematological malignancies expressing CD25, CD7, CD64, or CD33, respectively, agents targeting MCSP, for treating malignant melanomas, agents targeting a Lewis Y Antigen, for treating adenocarcinomas, and agents targeting IL13 or EGFR, for treating a variety of tumors known to express these antigens, such as glioblastomas. Methods for preparing immunological agents having a desired antigen specificity and, optionally, a toxin moiety conjugated thereto, can be prepared in accordance with the general methods disclosed above, and disclosed in Section II above, and specifically in Example 1 below for a CD19:ETA and MCSP:ETA conjugate.

Thus, an aspect of the invention involves identifying cancer patients who are candidates for effective anti-cancer treatment with an immunological agent, but for whom combined treatment with a sensitizing agent is desired to enhance the anti-tumor efficacy of the immunological agent.

In the preferred treatment method, the subject is administered the immunological agent in an amount that is effective inhibiting proliferation of cancer cells in the subject. The dose administered and the dosing schedule will follow, for example, known or recommended doses for antibody agents currently in use for anti-tumor therapy, such as Rituximab, as indicated, for example, in the drug product insert or published clinical or animal-model data. In the animal model methods described in Example 4A and 4B below, for example, the immunological agent was effective at a dose of 10 μg/20 g animal, or roughly 40 mg/80 kg human patient, although substantially lower doses, e.g., 1-20 mg for a human subject, are also contemplated. One advantage of the present invention is that lower-than-normal doses of the immunological agent may be administered, if necessary, due to the compensating enhancement effect of the sensitizing agent. Thus, a kit containing a dose of the immunological agent could optionally contain a product insert having one set of directions for using the agent in monotherapy, and another set of directions for using the agent in a combination therapy with the sensitizer. The set of instructions for the combination therapy could recommend a lower dose of the immunological agent, when used in combination with the sensitizer and/or a different dosing regimen for one or both agents, when used together, than would normally be recommended for the immunological agent when used alone.

The sensitizing agent may be administered, before, during, or after administration of the immunological agent. Typically, the two agents are administered in a common dosing regimen, as described below, and the two agents themselves may be administered in a combined-drug composition, e.g., by IV administration, or separately. However, a dosing regimen in which the sensitizer is administered before or after administering the immunological agent is also contemplated. For example, a person under treatment with an immunological agent may be subsequently placed on a combined therapy that includes the sensitizer.

Alternatively, the patient may be initially administered the sensitizer followed one-to-several days later with the immunological agent, In this regimen, the sensitizer functions, in part, to sensitize the cancer cells to inhibition by the immunological agent, by inhibiting mitochondrial function. Preferred dose levels and dosing schedules are considered further below.

The immunological agent may be administered by direct injection of a tumor or its vasculature. Alternatively, the tumor may be infused or perfused with the agents using any suitable delivery vehicle. The agents may be administered locally to an affected organ. Systemic administration may also be performed. Continuous administration may be applied where appropriate; for example, where a tumor is excised and the tumor bed is treated to eliminate residual disease. Delivery via syringe or catheterization is preferred. Such continuous perfusion may take place for a period from about 1-6 hours, to about 6-12 hours, to about 12-24 hours, to about 1-2 days, to about 1-2 weeks or longer following the initiation of treatment. Generally, the dose of the therapeutic composition via continuous perfusion will be equivalent to that given by a single or multiple injections, adjusted over a period of time during which the perfusion occurs.

The therapeutic agents are administered to a subject, such as a human patient, in a formulation and in an amount effective to achieve a clinically desirable result. For the treatment of cancer, desirable results include reduction in tumor mass (as determined by palpation or imaging; e.g., by radiography, radionucleotide scan, CAT scan, or MRI), reduction in the rate of tumor growth, reduction in the rate of metastasis formation (as determined e.g., by histochemical analysis of biopsy specimens), reduction in biochemical markers (including general markers such as ESR, and tumor specific markers such as serum PSA), and improvement in quality of life (as determined by clinical assessment, e.g., Karnofsky score), increased time to progression, disease-free survival and overall survival.

The amount of each agent per dose and the number of doses required to achieve such effects will vary depending on many factors including the disease indication, characteristics of the patient being treated and the mode of administration. Typically, the formulation and route of administration will provide a local concentration at the disease site of between 1 nM and 100 μM of each agent. The physician will be able to vary the amount of the agents, the carrier, the dosing frequency, and the like, taking into consideration such factors as the particular neoplastic disease state and its severity; the overall condition of the patient; the patient's age, sex, and weight; the mode of administration; the suitability of concurrently administering systemic anti-toxicity agents; monitoring of the patient's vital organ functions; and other factors typically monitored during cancer chemotherapy. In general, the compounds are administered at a concentration that affords effective results without causing excessive harmful or deleterious side effects.

Formulations. The pharmaceutical carrier(s) employed may be solid or liquid. Liquid carriers can be used in the preparation of solutions, emulsions, suspensions and pressurized compositions. The compounds are dissolved or suspended in a pharmaceutically acceptable liquid excipient. Suitable examples of liquid carriers for parenteral administration include water (which may contain additives, e.g., cellulose derivatives, preferably sodium carboxymethyl cellulose solution), phosphate buffered saline solution (PBS), alcohols (including monohydric alcohols and polyhydric alcohols, e.g., glycols) and their derivatives, and oils (e.g., fractionated coconut oil and arachis oil). The liquid carrier can contain other suitable pharmaceutical additives including, but not limited to, the following: solubilizers, suspending agents, emulsifiers, buffers, thickening agents, colors, viscosity regulators, preservatives, stabilizers and osmolarity regulators.

For parenteral administration, the carrier can also be an oily ester such as ethyl oleate and isopropyl myristate. Sterile carriers are useful in sterile liquid form compositions for parenteral administration. Sterile liquid pharmaceutical compositions, solutions or suspensions can be utilized by, for example, intraperitoneal injection, subcutaneous injection, intravenously, or topically. The compositions can also be administered intravascularly or via a vascular stent.

The liquid carrier for pressurized compositions can be a halogenated hydrocarbon or other pharmaceutically acceptable propellant. Such pressurized compositions may also be lipid encapsulated for delivery via inhalation. For administration by intranasal or intrabronchial inhalation or insufflation, the compositions may be formulated into an aqueous or partially aqueous solution, which can then be utilized in the form of an aerosol.

The compositions may be administered topically as a solution, cream, or lotion, by formulation with pharmaceutically acceptable vehicles containing the active compound. The compositions of this invention may be orally administered in any acceptable dosage including, but not limited to, formulations in capsules, tablets, powders or granules, and as suspensions or solutions in water or non-aqueous media. Pharmaceutical compositions and/or formulations comprising the oligonucleotides of the present invention may include carriers, lubricants, diluents, thickeners, flavoring agents, emulsifiers, dispersing aids or binders. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

The use of liposomes to facilitate cellular uptake is described, for example, in U.S. Pat. Nos. 4,897,355 and 4,394,448, and numerous publications describe the formulation and preparation of liposomes. Liposomal formulations can also be engineered, by attachment of targeting ligands to the liposomal surface, to target sites of neovascularization, such as tumor angiogenic regions. The compounds can also be formulated with additional penetration/transport enhancers, such as unconjugated forms of the lipid moieties described above, including fatty acids and their derivatives. Examples include oleic acid, lauric acid, capric acid, myristic acid, palmitic acid, stearic acid, linoleic acid, linolenic acid, dicaprate, tricaprate, recinleate, monoolein (a.k.a. 1-monooleoyl-rac-glycerol), dilaurin, caprylic acid, arichidonic acid, glyceryl 1-monocaprate, 1-dodecylazacycloheptan-2-one, acylcarnitines, acylcholines, mono- and di-glycerides and physiologically acceptable salts thereof (i.e., oleate, laurate, caprate, myristate, palmitate, stearate, linoleate, etc.). Other useful adjuvants include substrates for transendothelial migration, such as glucose uptake systems for facilitated egress from the vascular space to the tumor microenvironment.

### V. Measurement of Cell Proliferation

The anticancer activity of the therapeutic combinations can be evaluated using standard in vitro and in vivo assays. The ability of a composition to specifically inhibit the growth of tumor cells can be assayed using tumor cell lines in vitro, or in xenograft animal models in vivo. A preferred protocol for such growth curve assays is the short term cell viability assay described in Asai et al. (2003, cited above). In established xenograft models of human tumors, the test compound is administered either directly to the tumor site or systemically, and the growth of the tumor is followed by physical measurement. A preferred example of a suitable in vivo tumor xenograft assay is also described in Asai et al. (2003, cited above). Other examples are described in Scorski et al., Proc. Natl. Acad. Sci. USA, 94: 3966-3971 (1997) and Damm et al., EMBO J., 20:6958-6968 (2001).

The following examples illustrate methods for producing immunological agents effective to inhibit cancer-cell proliferation by inducing cancer-cell apoptosis, for demonstrating cancer-cell inhibition by the agents, and for inhibiting cancer cell proliferation in vitro and vivo in accordance with the methods of the invention. The examples are in no way intended to limit the scope of the invention.

### EXAMPLE 1

### Preparation of Immunological Agents

A. Construction of scFv CD19:ETA′

A single-chain Fv (scFv) antibody fragment reactive with human CD19 was generated by subcloning the hybridoma 4G7 (Meeker 1984). The cDNA coding for the scFv was fused to the coding sequence for truncated Pseudomonas Exotoxin A lacking the receptor-binding domain. The C-terminal pentapeptide REDLK that directs the retrograde transport of the wildtype bacterial toxin, was replaced by the coding sequence for the characteristic endoplasmic reticulum retention sequence KDEL. This replacement was performed following published examples to optimize intracellular transport to the ER (Seetharam 1991). Sequences coding for a STREP tag and a hexahistidine tag were added at the N-terminus for detection and purification.

The resulting polypeptide construct (FIG. 1A) was expressed in E. coli and purified from periplasmic extracts by affinity chromatography using a streptactin matrix. The CD19-immunotoxin (CD19:ETA′) specifically bound to the CD19-positive human B-cell precursor leukemia cell line Nalm-6. Binding was successively prevented by coincubation with increasing concentrations of the parental monoclonal antibody 4G7. CD19:ETA′ failed to bind to CD19-negative U937 cells, a cell line derived from a human monocytic leukaemia.

### B. Construction, Expression and Purification of the Recombinant Immunotoxin

The MCSP directed scFv was sub cloned from the hybridoma line 9.2.27 by phage display as described before (Peipp, M., Cancer Res., 62:2848-2855 (2002)) and fused to the coding sequence for truncated Pseudomonas ETA, containing domains II, Ib and III but lacking binding domain Ia. The coding sequence for the C-terminal REDLK-motiv was replaced by the coding sequence for the eukaryotic endoplasmatic reticulum retention motiv KDEL. This replacement improves the retrograde transport through the trans-golgi network and leads to an enhanced cytotoxicity of ETA′-derived immunotoxins (Seetharam, S., J Biol Chem, 266:17376-17381 (1991)). The variable light and heavy chain domains (V_{L} and V_{H}) were connected by a sequence coding for a 20 amino acid flexible linker (G₄S)₄. The same linker was used to connect the scFv moiety to the truncated ETA′. For purification and specific detection, sequences for an N-terminal STREP tag and a hexahistidine tag were added.

The resulting construct (FIG. 1B) was cloned into the bacterial expression vector pet27b and expressed in E. coli BL21 under osmotic stress conditions (Barth, S. et. al., Appl Environ Microbiol April 2000; 66:1572-1579). After a single purification cycle using Streptactin beads the recombinant immunotoxin was highly enriched. In Western transfer experiments the Immunotoxin reacts with antibodies specific for the ETA′-moiety and the hexahistidine tag, respectively. The yield was approximately 20-30 μg enriched recombinant protein per liter E. coli culture.

As evaluated by flow cytometric analyses, the MCSP-directed immunotoxin binds to human Melanoma cells M14-MCSP, stably transfected with MCSP c-DNA, whereas no binding occurs to the untransfected MCSP-negative M14 cells.

### EXAMPLE 2

### Cell Apoptosis Induced by Immunological Agents

### A. Dose-Dependent and Antigen-Restricted Induction of Apoptosis by CD19:ETA′

scFvCD19:ETA′ mediated specific death of CD19-positive Nalm-6 and Reh cells in a dose-dependent manner, but failed to eliminate CD19-negative CEM and U937 cells, as evidenced by measurement of nuclear DNA content after 72 h of treatment, using propidium iodide (PI) staining and flow cytometry. The effective concentration (EC₅₀) of CD19:ETA′ provoking a response half way between the baseline and maximum response of Nalm-6 cells was determined to be 175 ng/ml, corresponding to 2.5 nM.

To investigate whether cell death induced by CD19:ETA′ occured via apoptosis, cell death was measured by Annexin V and PI staining. Annexin V-positive, PI-negative early apoptotic cells were clearly detectable in CD19-positive cell lines Nalm-6, Reh and Namalwa after 48 h of single dose treatment. The cytotoxic effect was blocked by coincubation of the cells with 20-fold molar excess of the parental antibody (FIG.2A). In addition, treatment of Nalm-6, Reh and Namalwa cells with CD19:ETA′ for 24 h induced cleavage of poly(ADP-ribose) polymerase (PARP), which is a characteristic feature of apoptotic cells. Again, induction of apoptosis was prevented by adding 10-fold molar excess of the parental antibody (FIG.2B). In conclusion, CD19:ETA′ induces apoptosis in CD19-positive cell lines in a highly antigen-dependent manner and is effective in low nanomolar concentrations.

### B. Antigen Specific Induction of Apoptosis by MCSP:ETA′-KDEL

To evaluate whether cell death was attributable to apoptosis cells were specifically measured by Annexin V and PI double staining. Treated MCSP-positive A2058 (FIG. 3A) and A375M (FIG. 3B) cells display Annexin V positive and PI negative staining in flow cytometric analysis, which a represents cells in the early apoptotic stage. A2058 cells show 41% after 96 h in the early apoptotic stage, A375M display 35% after 72 h. Again, this cytotoxic effect could be blocked by pre-treatment with the parental antibody 9.2.27 in tenfold molar excess. A further prove of apoptotic cell death is the cleavage of poly ADP-ribose polymerase (PARP). MCSP:ETA′-KDEL induce the cleavage of intact PARP (116 kDa) to its characteristic 85 kDa proteolytic fragment in A2058 (FIG. 3C) and A375M (FIG. 3D) cells after 48 h of treatment. Thus MCSP:ETA′-KDEL specifically induce apoptosis in long term cultured human melanoma cells as shown by two independent methods.

### EXAMPLE 3

### Inhibition of Cancer-Cell Proliferation in vitro

A. Synergistic Cytotoxic Activity of CD19:ETA′ with Valproic Acid or Cyclosporin A

Cell lines Nalm-6, Reh and SEM were treated with either 100 ng/ml CD19:ETA′ and 100 μg/ml or 150 μg/ml VPA alone or with a combination of both agents for 72 h. Combination treatment resulted in significantly increased apoptosis induction in all tested cell lines (FIG.4A). To assess whether this effects were additive or even synergistic, the cooperativity index (C_{I}) was calculated. Whereas the effect on SEM cells was additive (C_{I}:1.0), cotreatment showed a synergistic effect towards Nalm-6 and Reh cells (C_{I}:0.9; 0.7).

To test whether CsA is also able to sensitize cells for cytotoxic effects of CD19:ETA′, Nalm-6, Reh and SEM cells were treated with the immunotoxin for 48 h in the presence or in the absence of CsA. Combination treatment with 100 ng/ml CD19:ETA′ and 6 or 10 μM CsA resulted in significantly higher induction of apoptosis as compared to single agent treatment in all tested cell lines (FIG.4B). The C_{I} for Nalm-6, Reh and SEM was calculated to be 0.4, 0.5 and 0.6, respectively, demonstrating a synergistic cytotoxic effect of CD19:ETA′ and CsA for all cell lines.

B. MCSP:ETA′-KDEL Cooperates with the Anti-Tumor Effect of CsA in a Synergistic Manner

Human melanoma cell line A2058 (FIG. 5A) were treated with varying concentrations of CsA as single agent or CsA in combination with a constant concentration of MCSP:ETA′-KDEL for 72 h and cell death was evaluated by PI staining of nuclei and flow cytometry. Due to the intracellular signaling properties of the MCSP antigen following antibody binding, A2058 cells were additionally treated with CsA in combination with the parental mAb 9.2.27 lacking a toxic moiety in equimolar concentration comparative to MCSP:ETA′-KDEL. As single agent CsA induces cell death in A2058 cells starting at concentrations between 10 and 25 μg/ml. In combination with 100 ng/ml MCSP:ETA′-KDEL cell death was synergistically enhanced, whereas no increase of cytotoxicity was measured with the mAb 9.2.27. The evaluated cooperative index (ci) for 1, 5 and 10 μg/ml CsA and 100 ng/ml MCSP:ETA′-KDEL ranges between 0.56 and 0.66 and indicates a strong synergistic cytotoxic effect of these two anti-tumor agents on long term melanoma cells lines. Synergistic cytotoxicity was also measured for melanoma cells from patient #4 (FIG. 5B). Therefore cells were incubated with CsA at 5 and 10 μg/ml as single agent or in combination with 1 μg/ml MCSP:ETA′-KDEL for 48 h. CsA single treatment displayed no cytotoxicity but CsA enhances the anti-tumor effect of MCSP:ETA-KDEL in a synergistic manner, indicated by a cooperative index of 0.65. Taken together, the cytotoxicity of MCSP:ETA′-KDEL and CsA synergizes in melanoma cells, as shown for long-term established melanoma cell-lines as well as for primary melanoma cell-strains.

### EXAMPLE 4

### Inhibition of Cancer-Cell Proliferation in vivo

In vivo Effects of CD19:ETA′ in NOD/SCID Mice Xenotransplanted with Nalm-6 Cells

In order to further verify the anti-leukemic effects of CD19:ETA′, the immunotoxin was evaluated in NOD/SCID mice xenotransplanted with Nalm-6 cells. For this purpose, 1×10⁶ cells were injected into the tail vein on day 0. Three days after tumor cell challenge a single dose of 10 μg of CD19:ETA′ or an irrelevant immunotoxin (CD7-ETA′; Peipp 2002) were injected i.v. and mice were observed for hind leg paralysis or loss of body weight>20%. Mice given CD19:ETA′ (n=11) survived significantly longer than mice treated with PBS (n=10, p=0.003) or the control immunotoxin (n=8, p=0.002; FIG. 6). Median survival time of the CD7-ETA′ control group was determined to be 30.5 days as compared to 73 days in the CD19:ETA′ treated group. These results indicate that CD19:ETA′ was able to significantly prolong survival in an aggressive leukemia model.

Although the invention has been described with respect to particular immunological agents, sensitizing agents, and treatment methods, it will be appreciated that various objects and features of the invention may be made without departing from the invention.

It is claimed:

1. A method for inhibiting the proliferation of cancer cells, comprising
(a) exposing the cells to an immunological agent capable of binding specifically to an antigen expressed on the surface of the cancer cells, in an amount of immunological agent, when given alone, that is effective to inhibit proliferation of cancer cells by inducing apoptosis in the cells, and
(b) either proceeding, following, or concomitantly with step (a), exposing the cells to a sensitizing agent selected from the group consisting of cyclosporin A and analogs thereof, and valproic acid and analogs thereof, in an amount of sensitizing agent effective to potentiate the anti-cancer effect of the immunologic agent, as evidenced by a level of inhibition of cancer-cell proliferation produced by exposing the cells to both agents that is supraadditive relative to the sum of the inhibitions of cancer-cell proliferation observed by exposing the cells to the immunological agent alone and to the sensitizing agent alone.

2. The method of claim 1, wherein the immunological agent is selected from the group consisting of an antibody alone, an antibody conjugated to a cell toxin, and an antibody conjugated to a small-molecule chemotherapeutic agent.

3. The method of claim 1, wherein the sensitizing agent is selected from the group consisting of cyclosporin A, NIM811, UNIL025, and PKF220-384.

4. The method of claim 1, wherein the sensitizing agent is valproic acid or 2-propyl-4-pentynoic acid.

5. The method of claim 1, for use in treating a cancer in a subject, wherein step (a) includes administering to the subject, an amount of the immunological agent that, when given alone, would be effective to inhibit proliferation of cancer cells in the subject, and step (b) includes administering to the subject, an amount of the sensitizing agent effective to potentiate the anti-cancer effect of the immunologic agent, as evidenced by a level of inhibition of cancer-cell proliferation produced by administering both agents to the subject that is supraadditive relative to the sum of the inhibitions of cancer-cell proliferation observed by administering the immunological agent alone and the sensitizing agent alone.

6. The method of claim 5, for treatment of a B-cell leukemia in a subject, wherein the immunologic agent includes an anti-CD19 antibody.

7. The method of claim 6, wherein the immunological agent is a conjugate of an anti-CD19 antibody and a protein toxin.

8. In a method for treating a subject with cancer, by administering to the subject, an immunological agent capable of binding specifically to an antigen expressed on the surface of cells of the cancer, to inhibit proliferation of the cells by inducing cell apoptosis, an improvement comprising
administering to the subject, before, during, or after administering the immunological agent, a sensitizing agent selected from the group consisting of cyclosporin A and analogs thereof, and valproic acid and analogs thereof, in an amount of the sensitizing agent effective to potentiate the anti-cancer effect of the immunologic agent, as evidenced by a level of inhibition of cancer-cell proliferation produced by administering both agents to the subject that is supraadditive relative to the sum of the inhibitions of cancer-cell proliferation observed by administering the immunological agent alone and the sensitizing agent alone.

9. The improvement of claim 8, wherein the immunological agent is selected from the group consisting of an antibody alone, an antibody conjugated to a cell toxin, and an antibody conjugated to a small-molecule chemotherapeutic agent.

10. The improvement of claim 8, for treatment of a B-cell leukemia in a subject, wherein the immunologic agent includes an anti-CD19 antibody.

11. The improvement of claim 10, wherein the immunological agent is a conjugate of an anti-CD19 antibody and a therapeutic moiety is a protein toxin.

12. The improvement of claim 8, wherein the sensitizing agent is selected from the group consisting of cyclosporin A, NIM811, UNIL025, and PKF220-384.

13. The improvement of claim 8, wherein the sensitizing agent is valproic acid or 2-propyl-4-pentynoic acid.

14. A kit for use in treating a cancer in a subject, comprising
(a) a dose of an immunological agent capable of binding specifically to an antigen expressed on the surface of cells of the cancer, effective in amount to inhibit proliferation of cancer cells in the subject by inducing cell apoptosis, and
(b) a dose of a sensitizing agent selected from the group consisting of cyclosporin A and analogs thereof, and valproic acid and analogs thereof, effective in amount to potentiate the anti-cancer effect of the immunologic agent, as evidenced by a level of inhibition of cancer-cell proliferation produced by administering both agents to the subject that is supraadditive relative to the sum of the inhibitions of cancer-cell proliferation observed by administering the immunological agent alone and the sensitizing agent alone.

15. The kit of claim 14, wherein the immunological agent is selected from the group consisting of an antibody alone, an antibody conjugated to a cell toxin, and an antibody conjugated to a small-molecule chemotherapeutic agent.

16. The kit of claim 14, for treatment of a B-cell leukemia in a subject, wherein the immunologic agent includes an anti-CD19 antibody.

17. The kit of claim 16, wherein the immunological agent is a conjugate of a CD19 antibody and protein toxin.

18. The kit of claim 14, wherein the sensitizing agent is selected from the group consisting of cyclosporin A, NIM811, UNIL025, and PKF220-384.

19. The kit of claim 14, wherein the sensitizing agent is valproic acid or 2-propyl-4-pentynoic acid.

20. A kit for use in treating a cancer in a subject, comprising
(a) an immunological agent that, when administered to the subject in a therapeutic dose, is effective to inhibit proliferation of cancer cells in the subject by inducing cell apoptosis, and
(b) a product insert having one set of directions for using the immunological agent in monotherapy, by administering the immunological agent to a subject at a therapeutic dose, and another set of directions for potentiating the anti-cancer effect of the immunological agent, when administered to the subject at a therapeutic dose, by administering to the subject, a sensitizing agent selected from the group consisting of cyclosporin A and analogs thereof, and valproic acid and analogs thereof, in amount of sensitizing agent effective to potentiate the anti-cancer effect of the immunologic agent, as evidenced by a level of inhibition of cancer-cell proliferation produced by administering both agents to the subject that is supraadditive relative to the sum of the inhibitions of cancer-cell proliferation observed by administering the immunological agent alone and the sensitizing agent alone.

21. The kit of claim 20, wherein the therapeutic dose of immunological agent to be administered, in accordance with the product insert, is less for the combination therapy than for the monotherapy.
